(19) United States
(12) Patent Application Publication
(10) Pub. No.: US 2008/0139460 A1
(43) Pub. Date: Jun. 12, 2008

(54) Binding agents for CD44 glycoproteins and methods of use

(75) Inventors: Lora Melnik, Rishon-LeZion (IL); Itshak Golan, Tzur-Hadassa (IL); David Naor, Jerusalem (IL); Yehiel Zick, Carmei Yosef (IL)

Correspondence Address: Martin D. Moynihan;PRTSI, Inc., P.O. Box 16446, Arlington, VA, 22215, US

(73) Assignees: Yissum Research Development Company of the Hebrew University of Jerusalem, Jerusalem (IL); Yeda Research And Development Co., Ltd., Rechovot (IL)

(21) Appl. No.: 11/907,893

(22) Filed: Oct. 18, 2007

Related U.S. Application Data
(62) Division of application No. 10/505,769, filed on May 11, 2005, now abandoned.
(60) Provisional application No. 60/360,003, filed on Feb. 28, 2002.

(51) Int. Cl. A61K 38/16; A61P 43/00

(52) U.S. Cl. 514/8

(57) ABSTRACT

The present invention is based on the finding that Gal-8 binds to carbohydrates of cell surface CD44std and CD44v and activates a chain of biological events within the cells. A specific effect exhibited was the induction of apoptosis in the CD44std and CD44v expressing cells.

Thus, the present invention concerns the use of an active agent for achieving a therapeutic effect on a target cell, the therapeutic effect comprises binding of said active agent to a standard CD44 (CD44std) glycoprotein or to a CD44 variant (CD44v) expressed by said target cell, the active agent being galectin-8 (Gal-8) or a functional derivative thereof. The active ingredient may be used for the preparation of a pharmaceutical composition, for the treatment of a disease or a disorder or for diagnostic purposes.

### RELATED APPLICATIONS

This application is a Divisional of U.S. patent application Ser. No. 10/505,769 filed on May 11, 2005, which is a National Stage of PCT Patent Application No. PCT/IL03/00153 filed on Feb. 27, 2003, which claims the benefit of U.S. Provisional Patent Application No. 60/360,003 filed on Feb. 28, 2002. The contents of the above Applications are all incorporated herein by reference.

### FIELD OF THE INVENTION

The present invention relates to ligands which bind to native as well as variants of the CD44 glycoprotein and to therapeutic effects achieved by said binding.

### LIST OF PRIOR ART

The following is a list of prior art which is considered to be pertinent for describing the state of the art in the field of the invention. Acknowledgement of these references herein will be made by indicating the number from their list below within brackets.
1. WO 95/15175
2. Hadari Y. R. et al. J. Cell Sci. 113, 2385-2397 (2000);
3. Naor, D., et al. Adv. Cancer Res. 71, 241-319, (1997);
4. Lesley, J., et al. Adv. Immunol. 54, 271-335, (1993);
5. Screaton, G. R., et al. J. Biol. Chem. 268, 12235-12238 (1993);
6. Van Weering, D. H. J., et al. PCR Methods Appl. 3, 100-106, (1993);
7. Lazebnik, Y. A., et al. Nature 371, 346-347 (1994);
8. Germain, M, et al. J. Biol. Chem. 274, 28379-28384 (1999);
9. Lesley, J., et al., J. Exp. Med. 182, 431-437, (1995);
10. Liu, F.-T. Clin. Immunol. 97, 79-88, (2000);
11. Bidon, N., Int. J. Mol. Med. 8, 245-250, (2001);
12. Gopalkrishnan, R. V., et al. Oncogene 19, 4405-4416, (2000);
13. Levy, Y, et al, J. Biol. Chem. 276, 31285-31295, (2001);
14. Hadari, Y. R., et al. Trends Glycoscie. & Glycotech. 9, 103-112, (1997);

### BACKGROUND OF THE INVENTION

### CD44 Glycoprotein

CD44 is a multi-structural glycoprotein involved in many physiological and pathological functions, including cell-cell and cell-matrix adhesion, support of cell migration, presentation of growth factors, chemokines or enzymes to corresponding cell surface receptors or relevant substrates, as well as transmission of signals from the membrane to the cytoskeleton or nucleus [Naor, D., et al. Adv. Cancer Res. 71, 241-319, (1997); Lesley, J., et al. Adv. Immunol. 54, 271-335, (1993)]. This glycoprotein is known to bind to multiple ligands (e.g. fibrinogen, fibronectin, alanine, collagen), the principal one being hyaluronic acid (HA).

The multi-structural nature of CD44 is generated by alternative splicing of nine (human) or ten (mouse) variant exons as well as by post-translational modifications [Screaton, G. R., et al. J. Biol. Chem. 268, 12235-12238 (1993)]. The alternative utilization of CD44 variant exons allows generation of hundreds of isoforms, which may restrictively be involved in distinct functions, discrete ligand binding and different pathological or physiological activities.

The isoform designation of CD44 is related to the order of alternatively spliced variants inserted into the membrane proximal domain of the CD44 extracellular region. Theoretically, hundreds of CD44 isoforms can be generated by alternative splicing [Van Weering, D. H. J., et al. PCR Methods Appl. 3, 100-106, (1993)] of 10 (mouse) or 9 (human) variant exons, designated V1 to V10, inserted in different combinations between the two constant regions consisting of 5 and 4 exons, at each end of the molecule. However, the number of CD44 variants (CD44v) identified so far is limited to a few dozen, detected mostly on epithelial cells, keratinocytes, activated leukocytes and many types of tumor cells [Naor, D., et al. (1997) ibid.]. For example, insertion of v3, v4, v5, v6, v7, v8, v9 and v10 variant exons between the constant regions of this molecule generates the CD44v3-v10 isoform (at times referred to by the name “keratinocyte CD44”), while insertion of v8, v9 and v10 variant exons generates the CD44v8-v10 isoform (at times, referred to by the name “epithelial CD44”). Direct splicing of constant exon 5 to constant exon 16 generates the standard CD44 (CD44s), which is much more abundant than the CD44 variants (CD44v). It was also found by reverse transcriptase polymerase reaction (RT-PCR) that synovial fluid cells from joints of rheumatoid arthritis (RA) patients predominantly express CD44v3-v10 isoforms (i.e., the keratinocyte isoform). However, nucleotide sequence analysis revealed that three extra bases (CAG) were illegitimately transcribed from the intron bridging exon v4 to exon v5, resulting in a new transcript (designated CD44vRA) translating CD44v3-v10 isoform with extra alanine between exon v4 and exon v5. The CD44vRA mRNA was isolated from RA synovial fluid cells reversed transcribed to cDNA, amplified by PCR, cloned in E. coli and then transfected into CD44-negative Namalwa cells. Namalwa cells expressing CD44vRA were designated Namalwa-CD44vRA cells. Using exactly the same procedure Namalwa-CD44v3-v10 cells were prepared (such cells included a CD44 identical to that of CD44vRA except for a missing alanine at the junction site between variant exon v4 and variant exon v5).

### CD44 Ligands

The multi-structural nature of CD44 may also influence its ligand repertoire. Indeed, CD44 has a wide range of ligands, the principal one being hyaluronic acid (HA, hyaluronate, hyaluronan), a linear polymer of repeating disaccharide units [D-glucuronic acid (1-β-3) N-acetyl-D-glucosamine (1-β-4)]ₙ. CD44 can, however, interact with several additional molecules such as collagen, fibronectin, fibrinogen, laminin, chondroitin sulfate, mucosal vascular addressin, serglycin/gp600, osteopontin (OPN) and the major histocompatibility complex class II invariant chain (li), as well as L-selectin and E-selectin [Lazebnik, Y. A., et al. Nature 371, 346-347 (1994); Germain, M, et al. J. Biol. Chem. 274, 28379-28384 (1999)]. In many cases CD44 does not bind to its ligand unless activated by external stimuli. As both CD44 and its ligand are ubiquitous molecules, this mechanism should avoid unnecessary engagement of the receptor. In fact, three states of CD44 activation have been identified in cell lines and normal cell populations [Lesley, J., et al., J. Exp. Med. 182, 431-437, (1995)]: active CD44, which constitutively binds HA; inducible CD44, which does not bind HA or binds it only weakly, unless activated by inducing monoclonal antibodies (mAbs), cytokines, growth factors or phorbol ester (Naor, D., et al, Clin. Lab. Sci. In Press.); and inactive CD44, which does not bind HA, even in the presence of inducing agents.

The involvement of CD44 in pathological activities may be confined not only to certain CD44 isoforms, but also to their interaction with specific ligands. This interaction may be dependent on the type of CD44 isoform or its post-translational modification (glycosylation and GAG attachments). Furthermore, the type of CD44 isoform may dictate the pattern of the post-translational modification. The rich ligand repertoire of CD44 is possibly related to its multistructural nature. Identification of existing as well as of novel CD44 ligands, especially those associated with pathological activities, may provide a new target for therapy. If the CD44 counter-molecule is preferentially engaged with cell surface CD44 involved in a pathological activity, a certain level of selective targeting of the ligand may be gained.

### Galectins and Galectin-8

Galectins are animal lectins that specifically bind β-galactoside residues [Liu, F.-T. Clin. Immunol. 97, 79-88, (2000)]. In mammals, 11 galectin members have been identified. Galectins are subdivided into three categories: (1) the prototype (galectins 1, 2, 5, 7 and 10) existing as monomers or homodimers consisting of one carbohydrate-recognition domain (CRD); (2) chimera type (galectin-3) containing a nonlectin part connected to a CRD; and (3) tandem repeat type (galectin 4, 6, 8, 9 and 12), comprising two distinct but homologous CRD's domains linked by a hinge peptide.

Galectins are detected in the cell nucleus and cell cytoplasm, as well as within the extracellular matrix [Bidon, N., Int. J. Mol. Med. 8, 245-250, (2001)]. While all galectins appear to interact with a number of glycoproteins, each individual galectin may recognize a discrete set. Some galectins recognize more than one glycoprotein, indicating either cross-specificity or recognition of different carbohydrate side chains on the same molecule. Galectins are associated with several biological mechanisms, including with components of the splicing machinery and with mRNA splicing, embryonic development, signal transduction, differentiation, transformation, tumor suppression, metastasis and the immune response, enhancement or inhibition of cell matrix interaction [Gopalkrishnan, R. V., et al. Oncogene 19, 4405-4416, (2000); Levy, Y, et al, J. Biol. Chem. 276, 31285-31295, (2001)].

Galectin-8, a member of the galectin family is a 34 kDa secreted β-galactoside-binding protein made up of two homologous (38% identity) carbohydrate-recognition domains (CRDs) linked by a short (26 amino acids) peptide [Levy, Y, et al, (2001), ibid.]. Galectin-8 is an integrin binding protein that interacts with several (α3, α6, and β1), but not all, members of the integrin family. Binding of galectin-8 to cells expressing the relevant integrins modulates their adhesion capacity and induces apoptosis upon them [Hadari, Y. R., et al. Trends Glycoscie. & Glycotech. 9, 103-112, (1997); Hadari, Y. R., et al. J. Cell Sci. 113, 2385-2397, (2000)].

Galectin-8 has been found in many malignant (lung, brain, prostate) [Bidon N., (2001), ibid.] and normal adult tissue (liver, brain, cardiac muscle, lung and kidney) [Hadari, Y. R., et al. (2000), ibid.]. Immobilized galectin-8 functions as a matrix protein, promoting cell adhesion by ligation and clustering cell surface integrin receptors [Levy, Y. et al. (2001), ibid.]. On the other hand, as a soluble ligand, galectin-8 forms a complex with integrins, delivering signals for inhibition of cell adhesion [Hadari Y. R. et al. (2000), ibid.]. Galectin-8 interacts with α3β1 and α6β1 integrins, but not with some other members of the integrin family. Immunoprecipation and immuno-histochemical analyses suggest that endogenous galectin-8, secreted from human carcinoma (1299) cells, forms complexes with cell surface α3β1 integrin. Soluble galectin-8 inhibits the adhesion of 1299 cells to plates coated with integrin ligands, and this effect can be reversed by Mn²⁺, which increases the affinity of integrins for their counter-molecules. The inhibitory effect of galectin-8 on integrin-dependent cell binding could not be mimicked by plant lectins or other galectins. Binding of galectin-8 to the cell surface of 1299 cells induced p53-independent apoptosis and its expression in these cells inhibited colony formation [Hadari Y. R. et al. (2000), ibid.]. In contrast, galectin-8 immobilized onto plastic or glutathione-S-tranferase (GST) promotes cell adhesion and cell spreading mediated by carbohydrates of cell surface integrins [Levy Y. et al. (2001), ibid.]. This sugar-protein interaction delivers a signaling cascade, which includes tyrosine phosphorylation of focal adhesion kinase and paxillin as well as activation of protein kinase B, p70S6 kinase and mitogen-activated protein kinase [Levy Y. et al. (2001), ibid.].

Cell adhesion to galectin-8 is potentiated by Mn⁺² and interrupted by soluble galectin-8, antibodies against integrin P1, EDTA, serum proteins and thiodigalactose (TDG), which inhibit the lectin-carbohydrate interaction. Immobilized monoclonal anti-galectin-8 antibodies also mediate cell adhesion. However, cell motility on immobilized galectin-8 takes place in the presence of serum, suggesting that partial detachment from the immobilized galectin-8 is necessary for cell migration. Truncated galectin-8 containing only one CRD largely lost its ability to modulate cell adhesion, implying that both CRD domains are required to maintain the activity of galectin-8.

### SUMMARY OF THE INVENTION

According to a first of its aspects the present invention concerns the use of a biologically active agent for the preparation of a pharmaceutical composition for achieving a therapeutic effect on a target cell, the therapeutic effect comprises binding of said active agent to a standard CD44 (CD44std) glycoprotein or to a CD44 variant (CD44v) glycoprotein expressed by said target cell, the active agent being galectin-8 (Gal-8) or a functional derivative thereof.

The term “biologically active agent” denotes an agent which has a regulatory or biochemical function on the target sites similar to that of the naturally occurring Gal-8, for example, can bind to the same cell-surface glycoprotein (or to another glycoprotein characterized by at least one identical epitope which binds and is activated by the Gal-8), can modulate cell-cell or cell-matrix adhesion, modulate cell migration, modulation signal transmission from cell surface to the cell cytoskeleton or nucleus or can induce programmed cell death, etc.

The term “therapeutic effect” for purposes herein is determined by such considerations as may be known in the art and should result in a measurable effect on a target cell. According to one embodiment, the therapeutic effect comprises induction of programmed cell death within the target cell.

The terms “standard CD44 (CD44std)” is used herein to denote the CD44 nucleic acid or amino acid in which the constant regions are substantially conserve, i.e. no alternative splicing has occurred in the original sequence. CD44std may be referred to herein at times by the term “original sequence”

“Alternative splicing” concerns the formation of variants of the original sequence as a result of alternative processing of the gene at the transcription level, as a result of different RNA editing at the post-transcriptional level.

Splice variants are sequences that occur naturally within the cells and tissues of individuals. The physiological activity of splice variant products and the original protein, from which they are varied, may be the same (although perhaps at a different level), opposite, or completely different and unrelated. In addition, variants may have no activity at all. When a variant and the original sequence have the same or opposite activity, they may differ in various properties not directly connected to biological activity, such as stability, clearance rate, tissue and cellular localization, temporal pattern of expression, up or down regulation mechanisms, and responses to agonists or antagonists.

According to the invention, the splice variants derived from the CD44 gene (i.e. from the original sequence) are designated “CD44 variant” or in short “CD44v”.

“Target cells” according to the invention include any cell which expresses and presents at least one isoform of the CD44 glycoprotein (CD44std and/or CD44v), and which is associated with a disease or disorder, such that the binding of the active agent to the CD44 glycoprotein, results in at least one biological event having a therapeutic effect with respect to said disease or disorder.

The term “binding” according to the invention concerns any type of physical association between the active agent and the CD44 glycoprotein (standard or variant), including engagement of one or more epitopdes by the active agent. The binding according to the invention results in the activation of the glycoprotein, thereby achieving a desired therapeutic/biological effect.

According to one embodiment, the active agent binds to CD44v via an epitope residing in the variant exon v6 present in CD44v. According to another embodiment, the active agent binds to CD44std via an epitope residing in the constant region of the glycoprotein.

Finally, the term “functional derivative thereof” used with respect to the active ingredient denotes an amino acid sequence having at least 70%, preferably 90% and more preferably 95% identity with Gal-8, when Gal-8 and the derivate are optimally aligned; and the functional derivative has substantially the same biological effect as Gal-8 on the target cells.

In another aspect, the invention concerns a method of achieving a biological effect on a target cell, the method comprising contacting said target cell with an effective amount of an active ingredient, the biological effect comprises binding of said active agent to CD44std or to CD44v expressed by said target cell, the active agent being Gal-8 or a functional derivative thereof.

The “biological effect” according to the invention involves any change in the regulatory or biochemical functions naturally occurring within the target cell, the change occurring as a result of binding of the active agent to CD44std or CD44v expressed by the cell and, as a result, activation of CD44std or CD44v. Preferably, the biological effect results in a therapeutic effect. The biological effect may include induction of apoptosis, (e.g. in hyper-proliferating cells, such as cancer cells; cells involved in autoimmune diseases, cells involved with chronic inflammatory diseases and/or cells engaged in allergic reactions).

According to yet another aspect, the invention concerns a pharmaceutical composition for achieving a therapeutic effect on a target cell, the therapeutic effect comprises binding of said active agent to CD44std or to a CD44v, the active agent being Gal-8 or a functional derivative thereof.

The therapeutic effect according to the invention is preferably correlated with the prevention or treatment of a disease. The term “prevention or treatment of a disease” according to the invention refers to the administering of a therapeutic amount of the active agent, the amount being effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The “effective amount” for purposes herein is determined by such considerations as may be known in the art of medicine. The amount must be effective to achieve the desired therapeutic effect, e.g. induction of apoptosis, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the active agent to the cell surface glycoprotein, its distribution profile within the body, a variety of pharmacological parameters such as half life in the body, on undesired side effects, if any, on factors such as age and gender, etc.

Finally, the present invention provides a method for identifying a subject having a disease or disorder associated with cells which comprise CD44std or CD44v glycoprotein, the method comprising:

(a) obtaining a biological sample from the subject;

(b) providing a probe comprising Gal-8 or a functional derivative thereof;

(c) contacting said biological sample with said probe under conditions which allow the binding of the probe to said CD44vRA to form a detectable probe-CD44vRA complex; and

(d) detecting probe-CD44vRA complexes, wherein the presence of said complexes indicates a high probability that the subject from which the sample was obtained has one of a disease or disorder involving cells which comprise the CD44std or CD44v glycoprotein.

As an alternative to step (d) above, the diagnostic method may comprise detecting probe-CD44vRA complexes and comparing the level of said complexes to the level of complexes detected in a second biological sample obtained from a healthy subject, a deviation from the level of complexes formed in the sample from the healthy subject indicating a high probability that the subject from which the first biological sample was obtained has one of a disease or disorder involving cells which comprise the CD44vRA molecule.

### BRIEF DESCRIPTION OF THE FIGURES

In order to understand the invention, a preferred embodiment will now be described, by way of non-limiting example only, with reference to the accompanying Figures, in which

FIGS. 1A-1C show flow cytometry analysis of the binding of Anti-C44std (FIG. 1A), anti-CD44v5 mAb (FIG. 1B) or anti-CD44v6 mAb (FIG. 1C) to Namalwa transfectants (Namalwa CD44 pcDNA3.1 (referred to as Namalwa), Namalwa CD44v3-v10, Namalwa CD44vRA or Namalwa std), in the absence (black curve, also marked by the digit “1”) or presence (dark gray curve, also marked by the digit “2”) of galectin-8; binding of a second antibody served as the control (light gray).

FIG. 2 shows a Western blot analysis with anti-pan CD44 mAb (Hermes 3) of the binding of extracts from Namalwa transfectants (pcDNA 3.1, Namalwa CD44v3-v10 or Namalwa CD44vRA cells) to immobilized glutathione-s-transferase (GST) (lane 2), to recombinant GST-galectin-8 fusion protein, in the absence or presence of tiodigalactoside (TDG) (lanes 3 and 4 respectively), total protein is represented by lane 1.

FIGS. 3A-3D are flow cytometry analysis of the binding of anti-CD44v6 mAb to the different Namalwa transfectants: Namalwa CD44 pcDNA3.1 (FIG. 3A), Namalwa CD44std (FIG. 3B), Namalwa CD44v3-v10 (FIG. 3C) or Namalwa CD44vRA (FIG. 3D)) in the absence (curve 1) or presence (curve 2) of galectin-8, and in the presence of lactose (curve 3) or in the presence of glucose (curve 4); the binding of a second antibody served as the control (curve 5).

FIGS. 4A-4B show the apoptotic effect of galectin-8 (Gal-8): FIG. 4A is a Western blot analysis with anti-PARP antibody, analyzing the apoptotic effect of Gal-8 on the different Namalwa transfectants wherein the ratio between the intensity of the lower band (the enzyme cleaved product) and the upper band (the intact product) indicates the strength of apoptosis; FIG. 4B is a bar graph presenting in this for each cell group, reflected as densocytometric signals, and divided by the ratio of signal obtained with none-treated cells. FCS and doxorubicin (Doxo) served as negative and position controls (respectively).

FIG. 5 is a Western blot analysis with anti-galectin-8 mAb showing the presence of several galectin-8 isoforms in synovial fluids derived from Rheumatoid Arthritis (RA) patients.

### DESCRIPTION OF THE INVENTION

The present invention is based on the novel finding that Gal-8 binds to carbohydrates of cell surface CD44std and CD44v and activates a chain of events within the cells, not occurring in the absence of the ligand. A specific effect exhibited was the induction of apoptosis in the CD44std and CD44v expressing cells.

Thus, according to a first of its aspects, the present invention concerns the use of an active agent for the preparation of a pharmaceutical composition for achieving a therapeutic effect on a target cell, the therapeutic effect comprises binding of said active agent to a standard CD44 (CD44std) glycoprotein or to a CD44 variant (CD44v) expressed by said target cell, the active agent being galectin-8 (Gal-8) or a functional derivative thereof. Gal-8 may be a naturally occurring agent (e.g. obtained from human, rat or mouse source etc.), semi-synthetic (e.g. a chemical modification of a naturally occurring molecule) or fully synthetic. According to one preferred embodiment, Gal-8 is derived from rat [JBC 270:73447-73453, (1995)]. According to yet another embodiment, Gal-8 is derived from human.

The CD44std or CD44v according to the invention are preferably expressed by the target cell and presented on its surface, i.e. as a cell surface glycoprotein.

Gal-8 has already been reported to bind to another, unrelated, cell surface glycoprotein, the integrin (WO 95/151751). Integrins are receptor proteins through which cells both bind to and respond to the extracellular matrix. They are part of a large family of cell adhesion receptors, which are involved in cell-extracellular matrix and cell-cell interactions. Functional integrins consist of two transmembrane glycoprotein subunits that are non-covalently bound. Those subunits are called alpha (α) and beta (β). The α-subunits all have some homology to each other, as do the β-subunits. The receptors always contain one alpha chain and one beta chain and are thus called heterodimeric and both of the subunits contribute to the binding of ligand.

CD44 and integrins differ both structurally and functionally and they belong to distinct families of proteins. Unlike integrins, CD44 is a single chain glycoprotein, which is a part of larger group, known as a link protein super-family. CD44, but not integrins, interact with carbohydrates-like molecules (hyaluronic acid) and with growth factors, using their heparin sulfate side chains, which are excluded from integrins.

According to one embodiment of the invention, CD44std glycoprotein is encoded by the nucleic acid sequence substantially as shown in SEQ ID NO:1 and the glycoprotein is comprised of the amino acid sequence substantially as detailed in SEQ ID NO:2. While the amino acid sequence presented in SEQ ID NO:2 is composed of the 20 naturally appearing amino acids, CD44std according to the invention may also refer to chemical modifications of the sequence depicted in SEQ ID NO:2. A “chemical modification” as used herein refers to a ‘CD44std-like’ glycoprotein which is not a splice variant of the glycoprotein presented in SEQ ID NO:2, however, in which at least one of its amino acids was modified either by natural processes (e.g. post-translational modifications) or by chemical modification techniques, which are all known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

According to one preferred embodiment, CD44std has the exact amino acid sequence as presented in SEQ ID NO:2.

According to yet another embodiment, the CD44 glycoprotein of the invention is an alternatively splicing variant of the glycoprotein depicted in SEQ ID NO:2. The CD44v of the invention may comprise one or more variant exons, preferably from the 10 (mouse) or 9 (human) variant exons already identified and designated V1 to V10, inserted in different combinations between the two constant regions of CD44std, and at one or both ends of said CD44std.

According to one preferred embodiment of the invention, the CD44v comprises the amino acid sequence substantially as shown in SEQ ID NO:4, and designated CD44vRA. A corresponding nucleic acid sequence is presented in SEQ ID NO:3.

According to another preferred embodiment, the CD44v of the invention comprises the amino acid sequence substantially as shown in SEQ ID NO:6, and designated CD44v3-v10. A corresponding nucleic acid sequence is presented in SEQ ID NO:5.

More preferably, the CD44v of the invention has one of the amino acid sequences shown in SEQ ID NO:4 or in SEQ ID NO:6.

The therapeutic effect according to the invention may comprise, without being limited thereto, inhibition of cell-cell or cell-matrix adhesion, inhibition of cell migration, modulation of signal transmission from the cell surface to the cell cytoskeleton or nucleus. Modulations of these biochemical functions may result, according to one aspect of the invention, in the induction of programmed cell death (apoptosis) of cells expressing said CD44std, or said CD44v.

One more specific embodiment of the invention concerns the use of an active agent for the preparation of a pharmaceutical composition for achieving a therapeutic effect on a target cell, the therapeutic effect comprising the binding of the active agent to a CD44vRA glycoprotein, the active agent being as defined above. The CD44vRA according to the invention comprises the amino acid sequence substantially as shown in SEQ ID NO:4, and more preferably, has the sequence shown in SEQ ID NO:4.

In any case, the therapeutic effect achieved by the present invention is correlated with the prevention or treatment of a disease or a disorder, the disease/disorder being associated with the expression and presentation of CD44std, CD44v or a combination of the same by the target cell. Non-limiting examples of diseases include cancer, autoimmune diseases, chronic inflammatory diseases and/or allergic reactions.

The invention also concerns a method of achieving a biological effect on a target cell, the method comprising contacting said target cell with an effective amount of an active ingredient, the biological effect comprises binding of said active agent to CD44std glycoprotein or to a CD44v expressed by said target cell, the active agent being Gal-8 or a functional derivative thereof.

The biological effect according to the invention preferably results in a therapeutic effect on the treated individual.

One preferred example of a therapeutic effect according to the invention concerns the binding and activation by Gal-8 of CD44vRA. It has been found that that synovial fluid cells from joints of rheumatoid arthritis (RA) patients predominantly express CD44vRA. Thus, the present invention may be of particular advantage in the treatment or prevention of RA, by administering to the subject having RA an effective amount of Gal-8 or a functional derivative thereof.

Yet further, the invention concerns pharmaceutical compositions for achieving a therapeutic effect on a target cell, the therapeutic effect comprises binding of said active agent to CD44std or to CD44v, the active agent being Gal-8 or a functional derivative thereof. The therapeutic effect, active agents and the CD44 glycoproteins are as defined above.

The active agent according to the present invention is administered and dosed in accordance with good medical practice, taking into account the clinical condition of the individual patient, the site and method of administration, scheduling of administration, patient age, sex, body weight and other factors known to medical practitioners.

The active agent of the invention may be administered in various ways. It should be noted that it can be administered alone or as an active agent in combination with pharmaceutically acceptable carriers, diluents, excipients, additives and adjuvants, as known in the art, e.g. to give form or consistency to the composition when it is given in a specific form, e.g. in pill form, as a simple syrup, aromatic powder, honey, and other various elixirs, to add flavors, colors, lubrication or the like to the composition. Other additives may be used for the purpose of enhancing uptake of the active agent by the cell or for enhancing the stability, sterility and isotonicity of the compositions. Such additives may include anti-microbial preservatives, antioxidants, chelating agents and buffers.

The pharmaceutically acceptable carrier/s, diluent/s, excipient/s, additive/s employed according to the invention are limited only by chemico-phyiso considerations, such as solubility and lack of reactivity with the active agent, and by the route of administration. The carrier/s, diluent/s, excipient/s, additive/s generally refer to inert, non-toxic solid or liquid fillers, diluents or encapsulating material and the like.

The active agent may be administered orally, subcutaneously or parenterally, including intravenous, intra-arterial, intramuscular, intraperitoneally and intranasal administration as well as by infusion techniques. While not specifically mentioned, other administration modes may be applied, according to the physicians' considerations.

Finally, the present invention provides a method for identifying a subject having a disease or disorder associated with cells which comprise (expressing and/or presenting) CD44std or CD44v glycoprotein, the method comprising:

(a) obtaining a biological sample from the subject;

(b) providing a probe comprising Gal-8 or a functional derivative thereof;

(c) contacting said biological sample with said probe under conditions which allow the binding of the probe to said CD44std or CD44v glycoprotein to form a detectable probe-CD44 complex; and

(d) detecting probe-CD44 complexes, wherein the presence of said complexes indicates a high probability that the subject from which the sample was obtained has one of a disease or disorder involving cells which comprise the CD44std or CD44v glycoprotein.

As an alternative to the above, the method may comprise detecting probe-CD44 complexes and comparing the level of said complexes to the level of complexes detected in a second biological sample obtained from a healthy subject, a deviation from the level of complexes formed in the sample from the healthy subject indicating a high probability that the subject from which the first biological sample was obtained has one of a disease or disorder involving cells which comprise the CD44std or CD44v glycoprotein.

The probe according to the invention may be anti-galectin-8 antibodies.

Detection of the probe-CD44 complexes can be carried out by any of a number of techniques well known in the art, including, without limitation those described in Sambrook et al. (1989).

### SPECIFIC EXAMPLES

### Materials

CD44-negative Namalwa Burkitt lymphoma cells were obtained from ATCC Manassas, Va.

Standard CD44 (CD44s) cDNA (SEQ ID NO:1) was obtained from Hela cervical cancer line.

CD44vRA cDNA (SEQ ID NO:3) was obtained from synovial fluid cells of RA patients.

CD44v3-v10 cDNA (SEQ ID NO:5) was obtained from human keratinocytes

Empty vector pcDNA3.1 was obtained from Invitrogen, Paisley.

Tiodigalactoside (TDG) was obtained from Sigma, Israel.

### Example 1

### Binding of Galectin-8 to Namalwa Cell Transfectants

### Methods

### Cell Transfection

To explore the capability of galectin-8 to interact with CD44 variants, CD44-negative Namalwa Burkitt Lymphoma cells were transfected with cDNA of CD44 variant (CD44v, i.e. CD44vRA and CD44v3-v10) or with CD44s, as well as with an empty vector (the latter being the control). Logarithmically growing Namalwa cells were harvested, washed with RPMI-1640 medium and re-suspended in RPMI medium containing 20% FCS. A quantity of 20 μg from the following expression vectors was added to 800 μl of cell suspension (4×10⁶ cells per ml): empty pcDNA3.1 vector, or pcDNA3.1CD44s, pcDNA3.1CD44v3-10 and pcDNA3.1CD44vRA to generate transfectants designated Namalwa pc3.1, Namalwa CD44s, Namalwa CD44v3-10, Namalwa CD44vRA cell respectively. Transfection was carried out by electroporation at 380 V and 500 μF, using a BIO-RAD, Gene Pulser. The cells were cultured in 6 ml LB medium containing 20% FCS, incubated for 24 h and then grown in the same medium supplemented with 0.5 mg/ml Zeomycin to select for the transfected cells. Transfected cell clones were obtained by limiting dilution and cDNA expression was confirmed by exon-specific RT-PCR, by using the specific primers.

### Western Blot Analysis

Cell extracts from the transfected cell lines were centrifuged for 15 minutes at 14,000 g at 4° C. and the supernatants (1 mg) were incubated at 4° C. for 2 hours with glutathion S transferase (GST) or with GST-galectin-8 (GST-rGal) immobilized on beads, in the presence or absence of tiodigalactoside (TDG). The beads were washed extensively and boiled and the bound proteins were resolved on 10% SDS-PAGE. The proteins were transferred to nitrocellulose and immunoblotted with Hermes anti-CD44s mAb.

### Flow Cytometry

The binding of galectin-8 to the various transfectants, in the presence or absence of anti-CD44v6 mAb, was also determined by flow cytometry. Briefly, Namalwa CD44v, Namalwa CD44s or Namalwa pcDNA3.1 cells were incubated with 3G5 anti-pan-CD44s (Hermes 3, IgG 1), anti-CD44v5 mAb or anti-CD44v6 mAb, in the presence or the absence of galectin-8. After 45 min on ice, the cells were washed extensively and incubated with fluorescein isothiocyanate (FITS)-conjugated secondary anti-Ig antibody for 30 min on ice. The cells were than washed and analyzed with a Flow Cytometry.

In another experiment the ability of lactose and glucose to interfere with the inhibition effect of galectin-8 on anti-CD44v6 mAb binding was analyzed. The flow cytometry analysis of anti-CD44v6 mAb binding to Namalwa transfectants incubated with galectin-8 was performed as described above, but in the presence or the absence of excess of lactose or glucose.

### Results

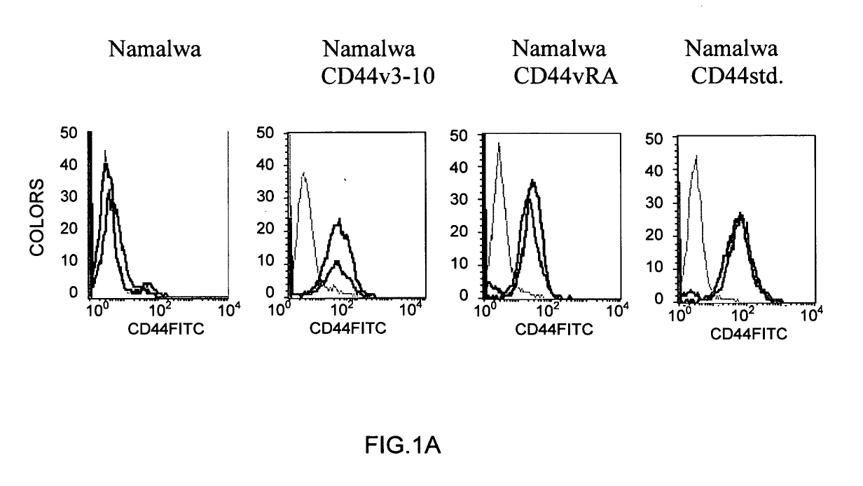

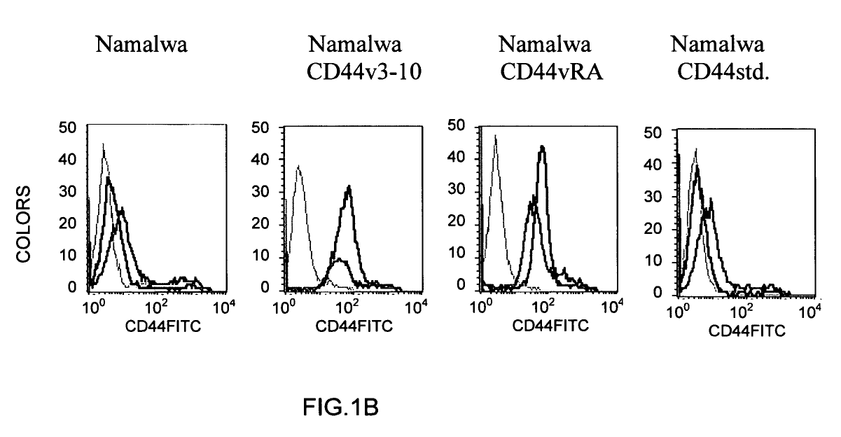

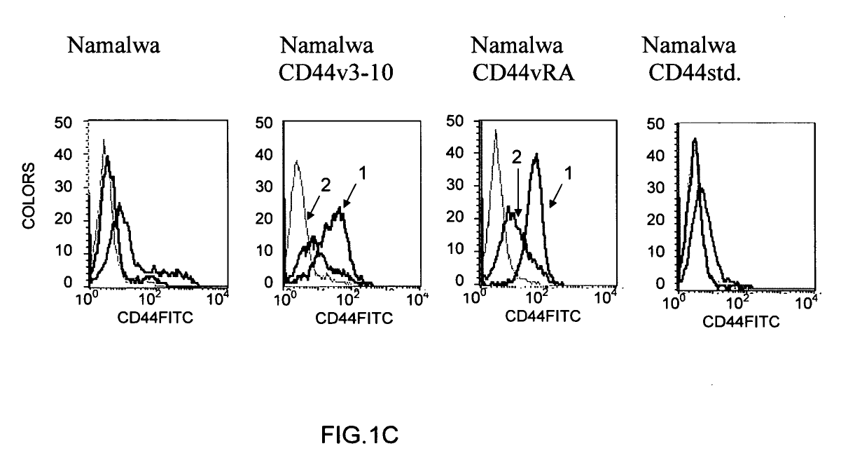

CD44s and CD44v were expressed to an equal extent on the Namalwa transfectants, as shown by flow cytometry (results are shown in FIGS. 1A-1C).

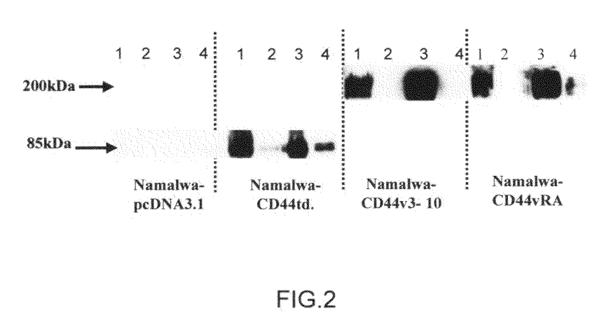

To evaluate the ability of CD44 from Namalwa transfectants to interact with galectin-8, extracts from these transfected cells were obtained. As shown by the Western blot analysis presented in FIG. 2, recombinant galectin-8 effectively bound almost the same extent to CD44 derived from Namalwa-CD44v and to that derived from Namalwa-CD44s while its ability to bind to extracts derived from Namalwa-pcDNA3.1 was not apparent (extracts from Namalwa cells transfected with empty vector were used as control). These findings suggest that galectin-8 bind to CD44 (variant as well as standard). The interaction with the protein was remarkably reduced in the presence of TDG.

In an additional experiment (FIGS. 1A-1C) it was shown that galectin-8 blocks the binding of anti-CD44v6 mAbs to Namalwa-CD44v cells (curve 2 in FIG. 1C), while the binding of anti-pan-CD44 (anti-CD44std) mAb or anti-CD44v5 mAb to Namalwa CD44v cells (FIGS. 1A and 1B, respectively) was not inhibited by Gal-8 (anti-pan-CD44 mAb recognizes a constant CD44 epitope shared by all CD44 isoforms). In addition, galectin-8 did not inhibit the binding of anti-pan-CD44 mAb to Namalwa-CD44s cells. These findings indicate that galectin-8 and the anti-CD44v6 mAb compete for the same binding site on the CD44 variant molecule. In contrast, galectin-8 does not compete with anti-CD44v5 mAb on the same binding site.

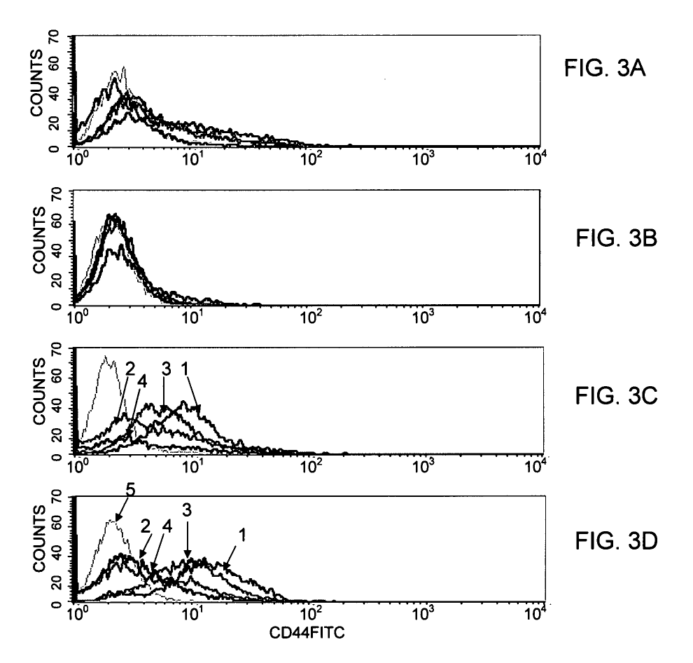

FIGS. 3A-3D show that the ability of galectin-8 to inhibit the binding of anti-CD44v6 to Namalwa CD44v cells was markedly reduced by the addition of lactose (curve 3), but not glucose (curve 4). This finding confirms that galectin-8 is bound to the lactose moiety of CD44v.

### Example 2

### Apoptosis Induction by Galectin-8

Poly(ADP-ribose) polymerase (PARP) is one of the caspase protein substrates cleaved during apoptosis by caspases-3 and 7 [Lazebnik, Y. A., et al. Nature 371, 346-347 (1994)]. The proteolytic cleavage of PARP, which synthesizes poly(ADP-ribose) from β-nicotinamide adenine dinucleotide (NAD) in response to DNA strand breaks, is an early biochemical event during apoptosis [Germain, M, et al. J. Biol. Chem. 274, 28379-28384 (1999)]. Since PARP cleavage is a hallmark of caspase activation, i.e., of apoptosis, the ability of galectin-8 to induce such biochemical activity in Namalwa-CD44-expressing cells was determined.

### Methods

The ability of galectin-8 to induce apoptosis in Namalwa-CD44-expressing cells was determined by Western blotting with anti-PARP antibody. To this end, the different Namalwa CD44 expressing cells were provided (as described in Example 1). The cells were treated as detailed Table 1 (cells with no treatment served as the negative control while Doxorubicin served as a positive control) Cells were grown in different treatment (serum free) media for 24 h with as indicated below. After incubation cells were lysed in 1% NP-40 lyses buffer and proteins were submitted to the 10% acril amid gel, transferred to the PVDF membrane and probed with anti-Poly(ADP-ribose) polymerase (PARP).

TABLE 1

| Experiment No. | Cell type | Treatment medium |
|---|---|---|
| 1 | Namalwa-CD44pcDNA3.1: | No treatment |
| 2 | | 10% FCS |
| 3 | | Galectin-8 2 μM |
| 4 | | Doxorubicin 5 μg/ml |
| 5 | Namalwa-CD44std: | No treatment |
| 6 | | 10% FCS |
| 7 | | Galectin-8 2 μM |
| 8 | | Doxorubicin 5 μg/ml |
| 9 | Namalwa CD44v3-v10: | No treatment |
| 10 | | 10% FCS |
| 11 | | Galectin-8 2 μM |
| 12 | | Doxorubicin 5 μg/ml |
| 13 | Namalwa CD44vRA: | No treatment |
| 14 | | 10% FCS |
| 15 | | Galectin-8 2 μM |
| 16 | | Doxorubicin 5 μg/ml |

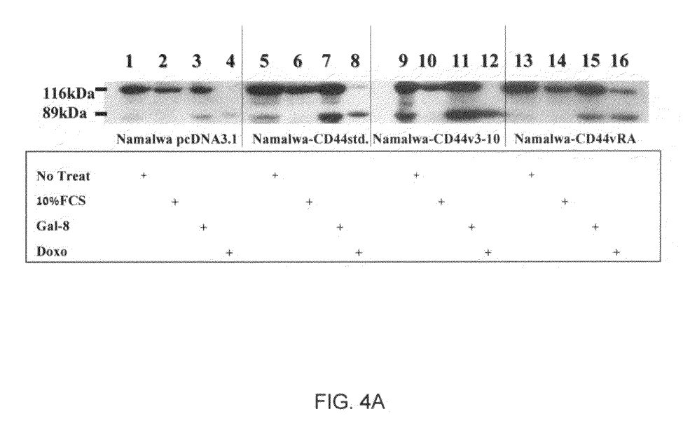

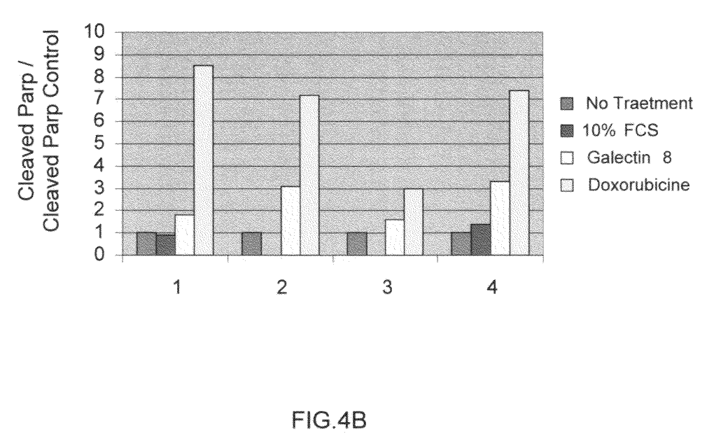

FIG. 4A presents the results of the different treatments, wherein the ratio between the intensity of the lower band (the enzyme cleavage product, cleaved PARP presented by the band of 85 kDa) and the upper band (the intact PARP, 116 kDa) indicates the strength of apoptosis. This ratio, reflected as densocytometric signals divided by the ratio of the negative control (no treatment) is presented in FIG. 4B. These results clearly show that in the presence of galectin-8 apoptosis is induced, with a less prominent effect on Namalwa CD44v3-V10 cells.

### Example 3

### Galectin-8 is Associated with Pathological Activities

### Methods

The presence of different galectin-8 isoforms in synovial fluid cells of RA patients was analyzed by the use of Western blot analysis of the synovial fluids and by the use of anti-galectin-8 antibody.

### Results

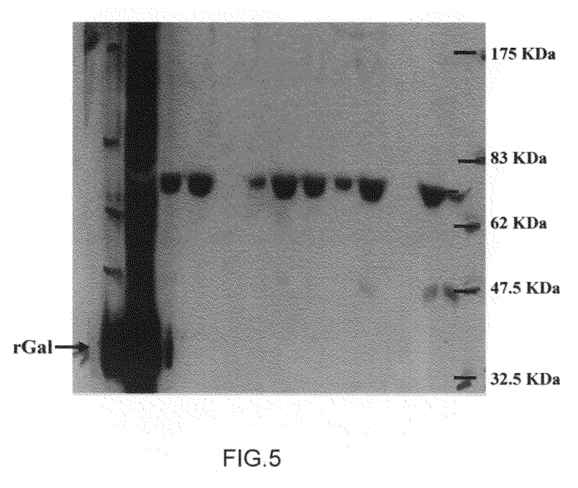

In order to investigate whether the interaction between cell surface CD44 and galectin-8 is associated with pathological activities, such as cell malignancy or destructive inflammation, the presence of different galectin-8 isoforms in synovial fluid cells of RA patients was analyzed. A Western blot analysis with anti-galectin-8 mAb is shown in FIG. 5. In particular, FIG. 5 shows the different galectin-8 isoforms detected in synovial fluids drawn from RA patients. This may suggest that galectin-8-CD44 interactions are confined to pathological isoforms.

1. A method of treating an inflammation in a subject, comprising administering to a subject a therapeutically effective amount of a galectin-8 polypeptide, thereby treating the inflammation in the subject.

2. The method of claim 1, wherein the inflammation involves cells expressing a CD44.

3. The method of claim 1, wherein the inflammation is a chronic inflammatory disease.

4. The method of claim 1, wherein the inflammation is rheumatoid arthritis (RA).

5. A method of inducing apoptosis in cells of a subject, comprising administering to the subject a therapeutically effective amount of a galectin-8 polypeptide, thereby inducing the apoptosis in cells of the subject.

6. The method of claim 5, wherein the cells express CD44vRA as set forth by SEQ ID NO:4.

7. The method of claim 5, wherein the cells are cancerous cells which express a CD44.

8. The method of claim 1, wherein said galectin-8 polypeptide is as set forth by SEQ ID NO:8.

9. The method of claim 5, wherein said galectin-8 polypeptide is as set forth by SEQ ID NO:8.


SEQUENCE LISTING (8 sequences)

SEQ ID NO: 1 - DNA, 1083 bp, Homo sapiens
atggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtg

SEQ ID NO: 2 - protein, 361 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 3 - DNA, 2100 bp, Homo sapiens
atggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgcagatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccagagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtacaacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtg

SEQ ID NO: 4 - protein, 700 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTTSSNTISAGWEPNEENEDERDRHLSFSGSGIDDDEDFISSTISTTPRAFDHTKQNQDWTQWNPSHSNPEVLLQTTTRMTADVDRNGTTAYEGNWNPEAHPPLIHHEHHEEEETPHSTSTIQATPSSTTEETATQKEQWFGNRWHEGYRQTPREDSHSTTGTAAASAHTSHPMQGRTTPSPEDSSWTDFFNPISHPMGRGHQAGRRMDMDSSHSTTLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 5 - DNA, 2097 bp, Homo sapiens
atggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccagagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtacaacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtg

SEQ ID NO: 6 - protein, 699 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPREDSHSTTGTAAASAHTSHPMQGRTTPSPEDSSWTDFFNPISHPMGRGHQAGRRMDMDSSHSTTLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 7 - DNA, 1247 bp, Rattus norvegicus
aattccccccctggctggggacaagttattactttgagtaatccttaaatgaagagtgggtaaagcccatatacggaagagagactccagtcaacaatatcaataagttgaagaagaaaaatgttgtccttaagcaatctacaaaatatcatctataacccgacaatcccctatgtcagtaccattactgagcagttgaagcctggctctttgatcgtgatccgtggccatgttcctaaagattcagaaagattccaagtagactttcagcatggcaacagcctgaagccgagagctgatgtggccttccactttaaccctcgcttcaaaaggtccaactgcattgtttgtaacacactgacaaatgagaaatggggctgggaggagatcacccacgacatgcctttcagaaaagaaaagtcctttgagattgtgatcatggtgctaaagaacaaattccacgtggctgtgaatggaaagcacattctgctgtatgcccacaggatcaacccagagaagatagacacactgggcatcttcggcaaagtgaacattcactccatcgggttcagattcagctcggatttacagagtatggaaacatctactctgggactgacacagataagtaaagaaaatatacaaaagtctggcaagctccatttgagcctgccatttgaagcaaggttgaatgcctccatgggccctggacgaaccgttgtcgttaaaggagaagtgaatacaaatgccacaagctttaatgttgacctagtggcaggaaggtcaagggatatcgctctgcacttgaacccacgcctgaatgtgaaagcgtttgtaagaaactcctttcttcaggatgcctggggagaagaggagagaaacattacctgcttcccatttagttctgggatgtactttgagatgataatttactgtgatgtccgagagttcaaggttgcagtaaatggtgtgcacagcctggagtacaagcacagatttaaagacctaagcagcatcgacacactagcagttgatggcgatatccgtttgctggatgtaaggagctggtagctatcatgactgccagaaccctggaaatacaaaatggcttatccgatactggccatgtcaaatgcatctcgctttcaccacattgttatactgttaagttgagctcgcacaacatcaagtcctactggtgttgtcaggcctggccatgcagtgtggctacctctgaattcccagga

SEQ ID NO: 8 - protein, 316 aa, Rattus norvegicus
MLSLSNLQNIIYNPTIPYVSTITEQLKPGSLIVIRGHVPKDSERFQVDFQHGNSLKPRADVAFHFNPRFKRSNCIVCNTLTNEKWGWEEITHDMPFRKEKSFEIVIMVLKNKFHVAVNGKHILLYAHRINPEKIDTLGIFGKVNIHSIGFRFSSDLQSMETSTLGLTQISKENIQKSGKLHLSLPFEARLNASMGPGRTVVVKGEVNTNATSFNVDLVAGRSRDIALHLNPRLNVKAFVRNSFLQDAWGEEERNITCFPFSSGMYFEMIIYCDVREFKVAVNGVHSLEYKHRFKDLSSIDTLAVDGDIRLLDVRSW